(19) United States
(12) Patent Application Publication
(10) Pub. No.: US 2025/0259735 A1
(43) Pub. Date: Aug. 14, 2025

(54) Systems and Methods for Preprocessing Medical Images

(71) Applicant: The Regents of the University of California, Oakland, CA (US)

(72) Inventors: Bahram Jalali, Los Angeles, CA (US); Callen MacPhee, Venice, CA (US); Yiming Zhou, Los Angeles, CA (US); Koichiro Kishima, Oakland, CA (US)

(73) Assignee: The Regents of the University of California, Oakland, CA (US)

(21) Appl. No.: 19/049,972

(22) Filed: Feb. 10, 2025

Related U.S. Application Data
(60) Provisional application No. 63/552,016, filed on Feb. 9, 2024.

(51) Int. Cl. G16H 30/40; G06N 5/04; G06N 20/00; G06T 5/60

(52) U.S. Cl. CPC G16H 30/40; G06N 5/04; G06N 20/00; G06T 5/60

(57) ABSTRACT

Systems and methods for preprocessing input images in accordance with embodiments of the invention are disclosed. One embodiment includes a method for performing inference based on input data, the method includes receiving a set of real-valued input images and preprocessing the set of real-valued input images by applying a virtual optical dispersion to the set of real-valued input images to produce a set of real-valued output images. The method further includes predicting, using a machine learning model, an output based on the set of real-valued output images, computing a loss based on the predicted output and a true output, and updating the machine learning model based on the loss.

### CROSS-REFERENCE TO RELATED APPLICATIONS

The current application claims the benefit of and priority under 35 U.S.C. § 119(e) to U.S. Provisional Patent Application No. 63/552,016 entitled “Method and System for Preprocessing Medical Images” filed Feb. 9, 2024, the disclosure of which is hereby incorporated by reference in its entirety for all purposes.

### FIELD OF THE INVENTION

The present invention generally relates to image processing and, more specifically, computer vision enhancement.

### BACKGROUND

Image enhancement refers to the process of improving the visual quality of an image by manipulating its attributes, such as brightness, contrast, and sharpness. Image enhancement techniques aim to produce images that are more visually appealing, informative, and useful for various applications. Image enhancement can be performed on digital images obtained from various sources such as cameras, satellites, and medical imaging devices. The need for image enhancement arises due to various factors, such as low lighting conditions, poor-quality sensors, and environmental factors that can affect the quality of the captured image.

Image enhancement techniques can be classified into two categories: point processing and spatial processing. Point processing techniques involve manipulating the pixel values of an image without considering the spatial relationship between neighboring pixels. Spatial processing techniques, on the other hand, use information from neighboring pixels to enhance the quality of the image. Image enhancement finds applications in numerous fields, such as medical imaging, satellite imaging, surveillance, and entertainment.

### SUMMARY OF THE INVENTION

Systems and methods for preprocessing input images in accordance with embodiments of the invention are disclosed. One embodiment includes a method for performing inference based on input data, the method includes receiving a set of real-valued input images and preprocessing the set of real-valued input images by applying a virtual optical dispersion to the set of real-valued input images to produce a set of real-valued output images. The method further includes predicting, using a machine learning model, an output based on the set of real-valued output images, computing a loss based on the predicted output and a true output, and updating the machine learning model based on the loss.

In another embodiment of the invention, preprocessing the set of real-valued input images includes transforming a set of real-valued input images to a set of complex-valued input images, applying a spectral phase kernel to the set of complex-valued input images, and converting the set of complex-valued input images to a set of real-valued intermediate output images. Preprocessing further includes determining an output phase based on the set of real-valued intermediate output images, and generating a set of real-valued output images by modifying the set of real-valued intermediate output images with the determined output phase.

In an additional embodiment of the invention, transforming the set of real-valued input images includes performing a 2D Fourier transform to project the real-valued input images into the spectral domain.

In yet another additional embodiment of the invention, applying a spectral phase kernel includes multiplying the set of complex-valued input images by a complex exponential, wherein the argument of the complex exponential includes a low-pass 2D function of frequency and a high-pass 2D function of frequency.

In still another additional embodiment of the invention, converting the set of complex-valued input images comprises performing a 2D inverse Fourier transform to project the set of complex-valued input images back into the spatial domain.

In yet still another additional embodiment of the invention, determining the output phase comprises computing an inverse tangent of the quotient of each pixel's imaginary component by each pixel's real component for the set of complex-valued input images.

In yet another embodiment of the invention, the output phase is determined using a Fourier differential theorem to approximate the output phase.

In still another embodiment of the invention, the set of real-valued input images comprises pathology images for cancer detection and tumor microenvironment analysis.

In yet still another embodiment of the invention, the spectral phase kernel comprises a Phase Stretch Transform (PST) algorithm for feature extraction and image processing in medical imaging applications.

In yet another additional embodiment of the invention, the PST algorithm preprocesses input images in the spatial domain by utilizing Fourier differentiation property.

In still another additional embodiment of the invention, the preprocessing comprises a Vision Enhancement via Virtual Diffraction and coherent Detection (VEViD) method for low-light enhancement and color-enhancement.

In yet still another additional embodiment of the invention, the method further includes updating the preprocessing based on the computed loss.

In yet another embodiment of the invention, the machine learning model comprises a convolutional neural network and a vision transformer.

In still another embodiment of the invention, the output phase indicates dispersion effects of the spectral phase kernel on the set of real-valued input images.

One embodiment includes a method for preprocessing input data, the method includes adding a constant DC bias term to each pixel of an input image to create a first modified image and multiplying each pixel of the first modified image by a negative constant gain value to create a second modified image. The method further includes dividing each pixel of the second modified image by a spatially corresponding pixel of the input image to create a third modified image and obtaining an output image by computing the inverse tangent of each pixel of the third modified image.

In yet still another embodiment of the invention, further including normalizing the output image by removing the DC component from the image data and equalizing the image.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates workflow for executing inference tasks with data preprocessed by PhyCV in accordance with an embodiment of the invention.

FIG. 2 illustrates a process for preprocessing input data for training and inference using the PhyCV algorithm in accordance with an embodiment of the invention.

FIG. 3 illustrates a generic training workflow for models that use data preprocessed by PhyCV for inference tasks in accordance with an embodiment of the invention.

FIG. 4 illustrates a training workflow of models that use data preprocessed by PhyCV for inference tasks with only model optimization in accordance with an embodiment of the invention.

FIG. 5 illustrates a joint optimization workflow of PhyCV and machine learning models in accordance with an embodiment of the invention.

FIG. 6 illustrates histological images of breast cancer obtained from five distinct hospitals within the Camelyon17-WILDS dataset and corresponding refined images using PhyCV in accordance with an embodiment of the invention.

FIG. 7 illustrates a comparison of neural network performance on the Camelyon17-WILDS dataset using various preprocessing techniques in accordance with an embodiment of the invention.

FIG. 8 illustrates a comparison of neural network workflows between Sobel and PhyCV preprocessing in accordance with an embodiment of the invention.

FIG. 9 illustrates a network architecture for performing training and inference tasks using input data preprocessed by PhyCV in accordance with an embodiment of the invention.

FIG. 10 illustrates a computing device that can be utilized to perform training and inference tasks using input data preprocessed by PhyCV in accordance with an embodiment of the invention.

FIG. 11 illustrates an edge server that can be utilized to perform training and inference tasks using input data preprocessed by PhyCV in accordance with an embodiment of the invention.

FIG. 12 illustrates an inference application for performing inference tasks using input data preprocessed by PhyCV in accordance with an embodiment of the invention.

### DETAILED DESCRIPTION

A persistent challenge in the realm of machine learning and artificial intelligence (AI), particularly in medical imaging, is managing the variability in data that can significantly impact the performance of learning algorithms. These variabilities may be caused by sample or subject preparation including biomarkers, differences between imaging machines, or variations in the parameters in images taken by the same machine. Traditional methods of preprocessing medical images often struggle to address issues such as variations in lighting, contrast, and noise, which can obscure critical diagnostic details.

This variability can pose a significant hurdle in developing robust machine learning models for classification. While deep learning has offered substantial improvements in image analysis, these models often require vast amounts of training data and are still susceptible to variations in image quality. Inconsistent training data can reduce model generalization, introduce bias, and lead to unreliable predictions. In medical applications, where precision and reliability are paramount, these challenges in model training may be magnified, and misinterpretation of medical images due to poor data quality can have serious implications for patient care.

This is where image enhancement technology can demonstrate its value. Image enhancement plays a crucial role in improving the visual quality of images, making them more visually appealing, informative, and useful for various applications. For example, when digital images are captured in environments with low light, they often suffer from undesirable visual qualities such as low contrast, loss of features, and poor signal-to-noise ratio. By altering image qualities such as brightness, contrast, and sharpness, image enhancement techniques can effectively enhance the visual quality of images. Image enhancement techniques play a crucial role in producing high-quality images that can be utilized in various fields, including but not limited to medical imaging, satellite imaging, surveillance, and entertainment.

Current methods for image enhancement are largely split between two categories of approaches: classical and deep-learning-based. Many classical algorithms involve the use of Retinex theory, which stems from concepts in human perception theory concerning the decomposition of an image into an illumination and a reflectance constituent. An example of a Retinex-based algorithm is LIME, which utilizes optimized Retinex theory for illumination map generation for high-quality enhancement. Other classical algorithms can include the histogram equalization method, which creates an expanded, more uniform histogram for contrast enhancement and increased dynamic range. However, histogram equalization methods often suffer from color distortion and other artifacts and may require additional processing and optimization.

Deep-learning-based image enhancement methods stem from the advancement of powerful data-driven machine-learning algorithms in recent years. Supervised learning methods such as LLNet, MBLLEN, EEMEFN, and TBEFN make use of ground truth datasets for training autoencoder-based algorithms. These methods are capable of high performance in target lighting conditions, but they can be limited in application to greater domains where training data are not readily available. The accuracy of loss functions can be affected by the absence of training data, which can make these methods less effective. Even if the lack of training data is overcome, these methods often do not clearly define the exact enhancement that is desired. This means that these algorithms may be able to produce enhancement effects that could satisfy the threshold set forth by the loss function but are still visually unsatisfactory. Moreover, the existing preprocessing techniques largely rely on general image enhancement methods, which may not account for the specific nuances and needs of medical imagery.

Deep neural networks (DNNs), known for their ability to handle large and complex datasets, are particularly suited for medical image analysis. While DNNs are powerful, they often struggle with overfitting the specific data distribution they are trained on. This overfitting can make it challenging for DNNs to generalize well to new, unseen data, especially when there are variations in environmental conditions such as contrast, brightness, and focus. There is a growing recognition of the need for more tailored and sophisticated preprocessing methods that can adapt to the unique characteristics of medical images and enhance their usability for machine learning models.

Systems and methods in accordance with many embodiments integrate physics-inspired principles into the preprocessing of medical images. Several embodiments utilize a novel preprocessing scheme, hereinafter referred to as PhyCV (Physics-inspired Computer Vision), to reduce non-semantic variability while preserving and even highlighting critical diagnostic information. Systems and methods in accordance with various embodiments can enhance the consistency, accuracy, and reliability of machine learning models in medical image analysis, leading to better patient outcomes and more efficient healthcare services.

Image enhancement techniques can be classified into two categories: point processing and spatial processing. Point processing techniques involve manipulating the pixel values of an image without considering the spatial relationship between neighboring pixels. Spatial processing techniques, on the other hand, use information from neighboring pixels to enhance the quality of the image. In numerous embodiments, systems and methods preprocess medical data using a set of algorithms that transform a real-valued input medical image into a complex-valued signal to improve the accuracy of automated medical imaging tasks. Transforming input images to complex-valued signals can address the inconsistencies related to current methods of training data generation, specifically across institutional gaps such as the collection environment and storage practices that might otherwise decrease machine learning efficacy.

PhyCV preprocessing in accordance with many embodiments complements neural networks by providing them with preprocessed images where irrelevant variabilities are reduced, allowing the deep layers of the network to learn and extract meaningful features relevant to medical diagnostics more effectively. By preprocessing the images to remove or reduce variability related to these environmental conditions, PhyCV can create a more uniform and consistent dataset. This uniformity is crucial because it means that the neural network is less likely to learn features specific to the training set's environmental conditions and more likely to learn the underlying, more general features relevant to the task at hand, be it diagnostic imaging in a medical context or another application. As a result, the use of PhyCV can enhance the neural network's ability to generalize to other data distributions, making it more robust and effective in real-world applications where data variability is a common challenge.

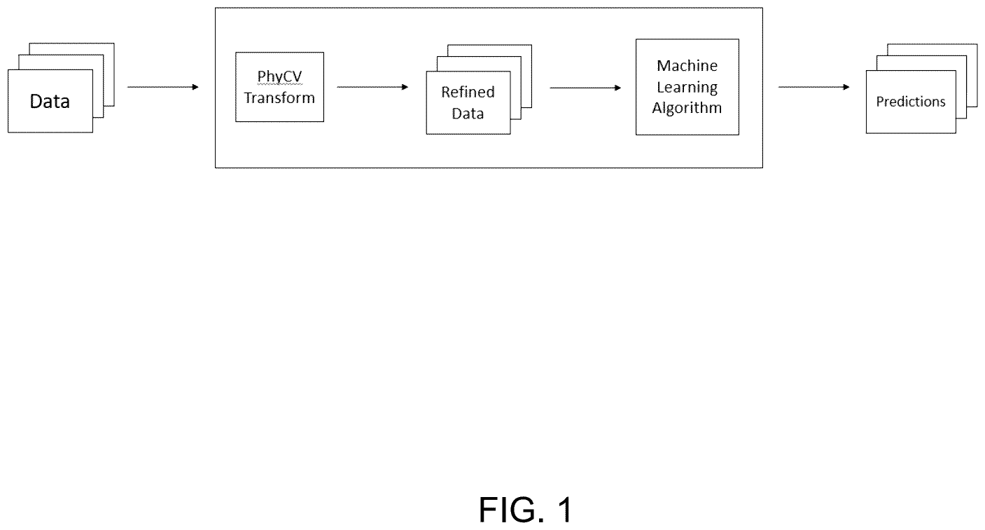

A workflow for executing inference tasks with data preprocessed by PhyCV in accordance with an embodiment is illustrated in FIG. 1. Input data, including images and videos, may undergo the PhyCV preprocessing process for refinement and feature normalization. Preprocessed input data in accordance with many embodiments are fed to machine learning algorithms to execute inference tasks. In various embodiments, inference pipelines generate predictive outputs based on the input data. PhyCV preprocessing may be a standalone process that is separate from the machine learning models that perform inference and predictions. In some embodiments, PhyCV preprocessing is incorporated into machine learning models as a single end-to-end package that refines input data for executing inference tasks.

In many embodiments, preprocessing systems convert data of input images to the frequency domain using a Fourier transform ( [symbol] ). Several embodiments apply a spectral phase kernel simulating virtual optical dispersion to the transformed input and revert the input data back to the original domain using an inverse Fourier transform ( [symbol] ⁻¹). Preprocessing systems in accordance with various embodiments perform coherent phase measurements on the reverted data and can further normalize the reverted data. In some embodiments, preprocessing systems utilize mathematical approximations to accelerate the preprocessing process and bypass the computationally costly Fourier and inverse Fourier transforms. Preprocessing systems in accordance with various embodiments output enhanced images to machine learning models for training and analysis. Systems and methods in accordance with numerous embodiments can greatly reduce data variability that does not represent useful semantic information for the training of analysis models, thereby enhancing the quality and reliability of medical data used in machine learning. Preprocessing systems may be utilized in both the training and inference stages of the machine learning process. In some embodiments, preprocessing systems can optimize itself during the course of training, specifically with respect to the optimization of the spectral phase kernel profile. Medical images of various types, including but not limited to pathology images, MRI, and X-ray data, may be refined using preprocessing systems, which will be discussed in further detail below.

### Data Refinement Via PhyCV

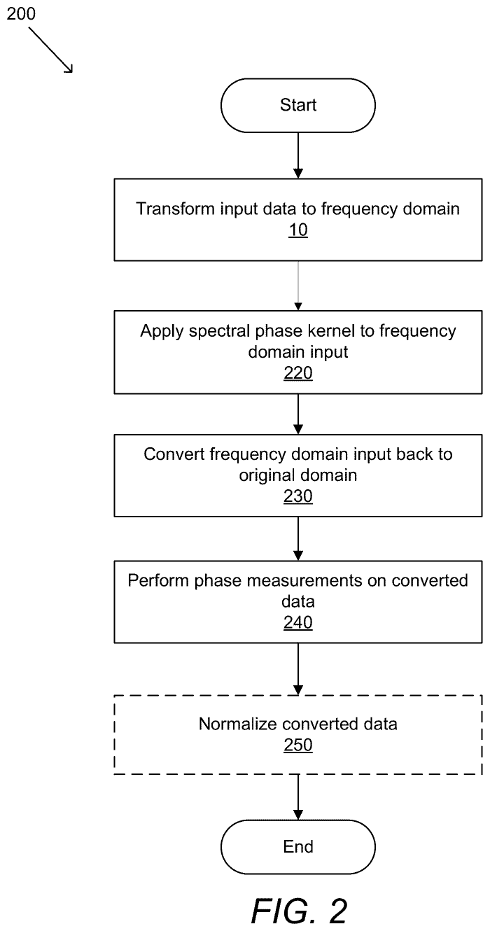

Preprocessing systems using PhyCV in accordance with many embodiments transform and preprocess input data to generate refined input data that facilitates better training and inference. A process for preprocessing input data for training and inference using the PhyCV algorithm in accordance with an embodiment of the invention is illustrated in FIG. 2. Process 200 transforms (210) input data into the frequency domain to obtain frequency-domain versions of the input. Several embodiments utilize the frequency-domain version of the input to identify frequency components that contribute to data variability in the input and manipulate the identified frequency components to control data variability in the input. Input data in accordance with many embodiments include images and videos. In some embodiments, Fourier Transforms can be implemented using the Fast Fourier Transform (FFT).

Systems and methods in accordance with many embodiments apply (220) a spectral phase kernel to the input data in the frequency domain. Many embodiments apply spectral phase kernels to simulate the application of virtual optical dispersions.

Process 200 converts (230) dispersed frequency domain input data back. In various embodiments, preprocessing systems apply an inverse Fourier Transform ( [symbol] ⁻¹) to transform the data back to its original domain. Converting back to the original domain can provide a format that is suitable for interpretation and further processing.

Process 200 performs (240) phase measurements on the converted data. Various embodiments capture and quantify phase alterations introduced by the application of the spectral phase kernel, which can provide insights into the dispersion effects on the data. Detecting and quantifying the computational phase alteration can correspond to coherent detection in a physical system.

Preprocessing systems may optionally normalize (250) the converted data to standardize the intensity levels across the dataset. Many embodiments perform this normalization to maintain consistency, especially when dealing with medical data from various sources and modalities. Data preprocessed by PhyCV may be used as input for machine learning algorithms, particularly deep neural networks. By preprocessing the data using the PhyCV transform, machine learning models are fed with data having reduced non-semantic variability, thereby potentially improving learning efficiency and diagnostic accuracy.

While specific processes for preprocessing input data for training and inference using the PhyCV algorithm are described above, any of a variety of processes can be utilized to preprocess input data for training and inference as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

Combination of PhyCV with Machine Learning

Systems and methods in accordance with many embodiments utilize PhyCV to preprocess input data to refine the input for more accurate inference. Many embodiments integrate PhyCV preprocessing with deep neural networks in both training and inference phases, which can represent a significant advancement in the application of machine learning to medical imaging, potentially leading to more accurate diagnoses and effective treatments.

During the training of models, deep neural networks can learn to recognize and interpret patterns in medical images. PhyCV preprocessing may be applied to each training image such that the network trains on data where non-semantic variability has been minimized. This preprocessing can enhance the neural network's ability to focus on relevant features, improving its learning efficiency. The spectral phase kernel within the PhyCV process can be optimized during training. By analyzing the neural network's performance on a validation set, adjustments to the kernel can be made to further reduce data variability and improve model accuracy.

In the inference phase, the trained models may be used to analyze new medical images. In various embodiments, PhyCV preprocessing is again applied to each image before it is fed into the model. This can provide consistency between the training and inference processes and allows the model to reliably interpret and diagnose new images based on the patterns it learned during training.

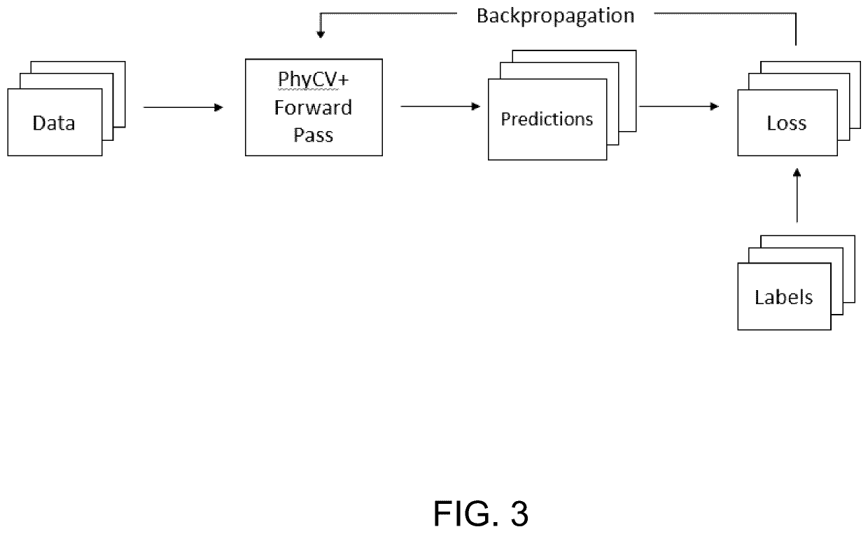

A generic training workflow for models that use data preprocessed by PhyCV for inference tasks in accordance with an embodiment is illustrated in FIG. 3. In many embodiments, models undergo the workflow illustrated in FIG. 1 to generate predictive outputs. Generated predictive outputs in accordance with various embodiments are evaluated against true labels under a supervised learning setting to compute the loss of the models that performed the inferences. Loss may be utilized to inform the backpropagation process to update the models to generate better predictions, leading to the optimization and enhancement of the models' parameters for improved performance.

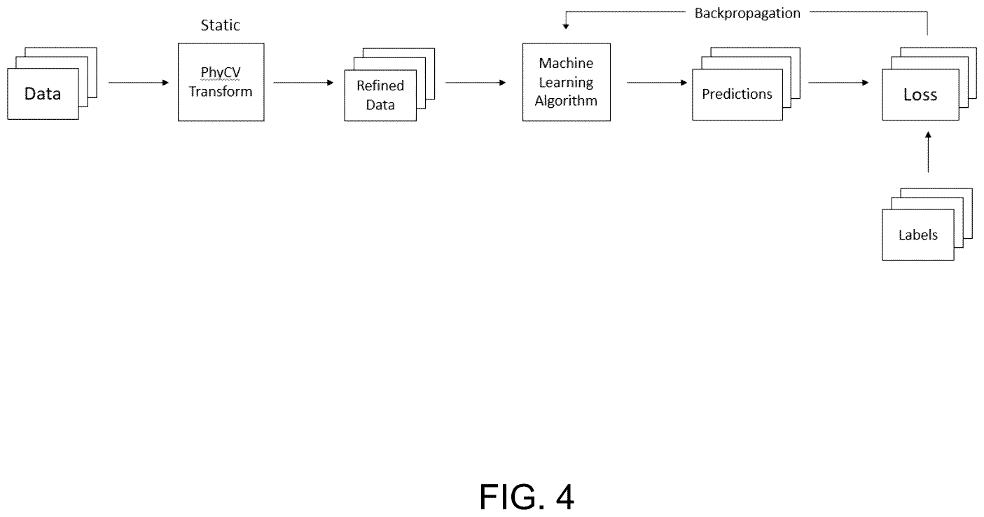

A training workflow of models that use data preprocessed by PhyCV for inference tasks with only model optimization in accordance with an embodiment is illustrated in FIG. 4. In several embodiments, PhyCV preprocessing may remain constant to serve as a fixed preprocessing step for the input data. Various embodiments generate predictions using machine learning models with the preprocessed input data, and the generated predictions are evaluated against actual labels to determine loss. Losses determined may be used only to update the parameters of the machine learning models, while PhyCV remains unchanged. In many embodiments, machine learning models' predictive accuracy can be fine-tuned by optimizing their parameters within the stable context provided by the unchanged PhyCV transformations.

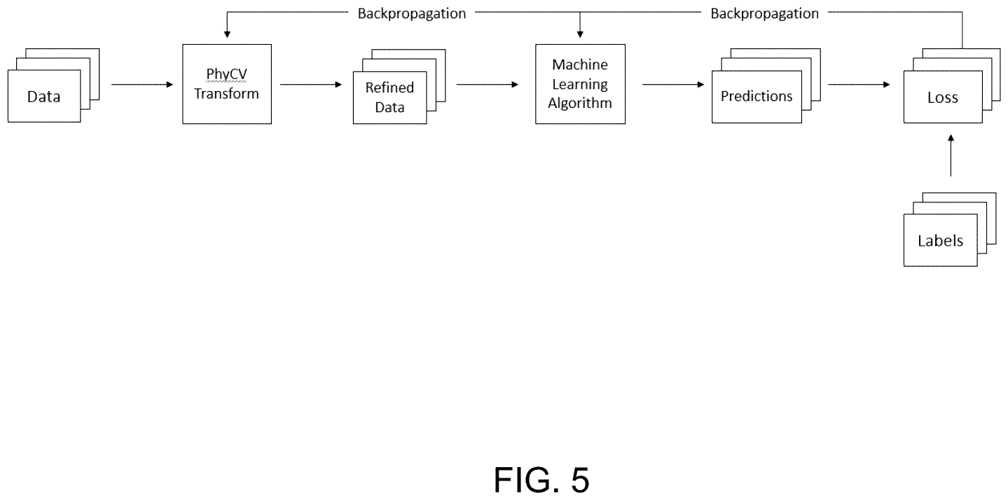

In some embodiments, PhyCV preprocessing may also be fine-tuned together with the associated machine learning model for inference tasks. A joint optimization workflow of PhyCV and machine learning models in accordance with an embodiment is illustrated in FIG. 5. Various embodiments enable both PhyCV and the machine learning model parameters to be fine-tuned concurrently. Input data may be preprocessed by PhyCV and used to generate predictive outputs. Loss may be determined in a manner like the manner described above by evaluating the generated predictions against actual labels. In many embodiments, the computed loss is used to inform the backpropagation process for both the model and the PhyCV preprocessing process. Specifically, the profile of the spectral phase kernel can be fine-tuned based on the training data and learning feedback, allowing for adaptive preprocessing that is closely aligned with the specific requirements of the medical data tasks. PhyCV can better preprocess data based on the feedback provided by the backpropagation process, and this integrated optimization approach can enhance the overall predictive performance by dynamically adjusting the preprocessing and learning components.

### Physics and Mathematics Foundation

PhyCV in accordance with many embodiments takes advantage of a process in which a real-valued input image becomes a complex-valued output image upon electromagnetic propagation through a thin virtual dielectric, and the phase of the complex-valued image is used as the output of the preprocessing step. While human eyes and common image sensors respond to the power in the light, electromagnetic diffraction optical imaging systems can work with both the intensity and phase of light, with the latter being measured through coherent detection. In many embodiments, PhyCV systems can discretize digital images to the 2D spatial domain to present images as spatially varying light fields. PhyCV systems can subject the field to physical processes akin to diffraction and coherent detection but in a virtual fashion. In various embodiments, light fields may be pixelated, and propagation of the light fields can impart a phase with an arbitrary dependence on frequency, which can be different from the monotonically increasing behavior of physical paraxial diffraction. Systems and methods in accordance with many embodiments execute processes based on physics and mathematics calculations similar to the calculations described in PCT Application No. PCT/US2023/077807 entitled “Systems and Methods for Vision Enhancement Via Virtual Diffraction and Coherent detection”, filed Oct. 25, 2023, the disclosure of which is incorporated by reference herein in its entirety.

A general solution to the homogeneous electromagnetic wave equation in rectangular coordinates (x, y, z) can be presented as:

$${E}_{o} ( x , y , z ) = {\int }_{- ∞}^{+ ∞} {\int }_{- ∞}^{+ ∞} {\tilde{E}}_{i} ( {k}_{x} , {k}_{y} , 0 ) {e}^{+ i {k}_{z} z} {e}^{i ( {k}_{x} x + {k}_{y} y )} {dk}_{x} {dk}_{y} \qquad ( 1 )$$

where \tilde{E}ᵢ(kₓ, k_{y}, 0) is the spatial spectrum of the input field Eᵢ(x, y, 0). Then, the Fourier content of the signal after a distance z can gain a phase term which can be represented by a spectral phase, ϕ(kₓ, k_{y}),

$${\tilde{E}}_{o} ( {k}_{x} , {k}_{y} , z ) = {\tilde{E}}_{i} ( {k}_{x} , {k}_{y} , 0 ) {e}^{- i ϕ ( {k}_{x} , {k}_{y} )} . \qquad ( 2 )$$

The phase represents the total accumulated over the propagation length. The forward propagated signal subjected to diffractive phase may be rewritten as,

$${E}_{o} ( x , y , z ) = {ℱ}^{- 1} \{ {\tilde{E}}_{i} ( {k}_{x} , {k}_{y} , 0 ) {e}^{- i ϕ ( {k}_{x} , {k}_{y} )} \} , \qquad ( 3 )$$

where [symbol] ⁻¹ refers to the inverse Fourier transform. E(x, y, z) now contains a frequency-dependent phase profile that is entirely described by the arbitrary phase ϕ(kₓ, k_{y}). The propagation can convert a real-valued input Eᵢ(x, y, 0) to a complex function Eₒ(x, y, z).

The above analysis can be translated from a continuous-valued E(x, y) in the spatial domain to discrete waveform E[n, m] for digital images. Similarly, analysis of the continuous momentum (kₓ, k_{y}) in the frequency domain may be performed on discrete momentum [kₘ, kₘ].

Light fields can be defined as the distribution of “field” strength across a two-dimensional landscape of the input signal with the pixel brightness mapped onto the field strength. The equivalent temporal frequency of a light field may have three bands corresponding to the three fundamental color channels (RGB). To obtain light fields of color images, in many embodiments, input RGB images are transformed into the HSV color space. Input image transformed into the HSV color space may be denoted as E[n, m; c], where c is the index for the color channel. To preserve color integrity, the diffractive transformation may be operated only on the “V” channel of the image when performing low-light enhancement.

Eᵢ[n, m; c], which represents an image frame, can be conceptualized as an information-carrying “pulse” that is subjected to diffraction, producing a complex output Eₒ[n, m; c], before applying coherent detection to extract the phase of the output. A normalization process [symbol] (⋅) may be performed to map “phixel” values to the appropriate range for digital image representation. Mathematically, this is formulated as,

$${E}_{o} [ n , m ; c ] = {ℱ}^{- 1} \{ ℱ \{ {E}_{i} [ n , m ; c ] + b \} \cdot H [ {k}_{n} , {k}_{m} ] \} . \qquad ( 4 )$$

[symbol] and [symbol] ⁻¹ refer to the 2D Fourier transform and inverse Fourier transform, respectively, that may be performed on input signals. b is a regularization term, and G is the inverse gain term. The function tan⁻¹(⋅) can calculate the phase pixel, which is referred to “phixel,” as discussed above. In selected embodiments, the spectral phase filter kernel H[kₙ, kₘ] has a phase profile ϕ[kₙ, kₘ] that comes with a low-pass characteristic.

When the spectral phase filter ϕ[kₙ, kₘ] has a high-pass characteristic, and the phase is small. The above process can be approximated using the Fourier differential theorem. Let ϕ[kₙ, kₘ] be the quadratic phase function and assume S<<1, then small phase approximation may be utilized:

$$H [ {k}_{n} , {k}_{m} ] = exp ( - i \cdot S ( {k}_{n}^{2} + {k}_{m}^{2} ) ) \approx 1 - i \cdot S ( {k}_{n}^{2} + {k}_{m}^{2} )$$

$$\begin{matrix}{E}_{o} [ n , m ] & = {ℱ}^{- 1} \{ ℱ \{ {E}_{i} [ n , m ] \} \cdot H [ {k}_{n} , {k}_{m} ] \} \\ & = {ℱ}^{- 1} \{ {\tilde{E}}_{i} [ {k}_{n} , {k}_{m} ] \cdot ( 1 - i \cdot S ( {k}_{n}^{2} + {k}_{m}^{2} ) ) \} \\ & = {ℱ}^{- 1} \{ {\tilde{E}}_{i} [ {k}_{n} , {k}_{m} ] - iS \cdot {\tilde{E}}_{i} [ {k}_{n} , {k}_{m} ] \cdot ( {k}_{n}^{2} + {k}_{m}^{2} ) \} \\ & = {E}_{i} [ n , m ] - i \cdot S \cdot \frac{{\partial }^{2} {E}_{i} [ n , m ]}{\partial {n}^{2}} + \frac{{\partial }^{2} {E}_{i} [ n , m ]}{\partial {m}^{2}} .\end{matrix}$$

The detected phase may be represented as:

$${tan}^{- 1} ( \frac{Im \{ {E}_{o} [ n , m ] \}}{Re \{ {E}_{o} [ n , m ] \}} ) = {tan}^{- 1} \frac{- S \cdot ( \frac{{\partial }^{2} {E}_{i} [ n , m ]}{\partial {n}^{2}} + \frac{{\partial }^{2} {E}_{i} [ n , m ]}{\partial {m}^{2}} )}{{E}_{i} [ n , m ]}$$

In this way, the Fourier transforms are eliminated, and the computations remain exclusively in the spatial domain.

When the spectral phase filter has a constant phase, and the phase is small. The above process can be approximated by the following derivation:

$$H [ {k}_{n} , {k}_{m} ] = exp ( - i \cdot S ) \approx 1 - i \cdot S .$$

Then the imaginary term of the output Eₒ[n, m; c] becomes:

$$\begin{matrix}Im \{ {E}_{o} [ n , m ; c ] \} & = Im \{ {ℱ}^{- 1} \{ ℱ \{ {E}_{i} [ n , m ; c ] + b \} \cdot exp ( - i ϕ [ {k}_{n} , {k}_{m} ] ) \} \} \\ & = Im \{ {ℱ}^{- 1} \{ ℱ \{ {E}_{i} [ n , m ; c ] + b \} \cdot ( 1 - i \cdot S ) \} \} \\ & = - S \cdot ( {E}_{i} [ n , m ; c ] + b ) ,\end{matrix}$$

where the detected phase will be:

$${tan}^{- 1} ( - S \cdot \frac{{E}_{i} [ n , m ; c ] + b}{{E}_{i} [ n , m ]} )$$

In this way, the Fourier transforms are eliminated, and the computations remain exclusively in the spatial domain.

### Results

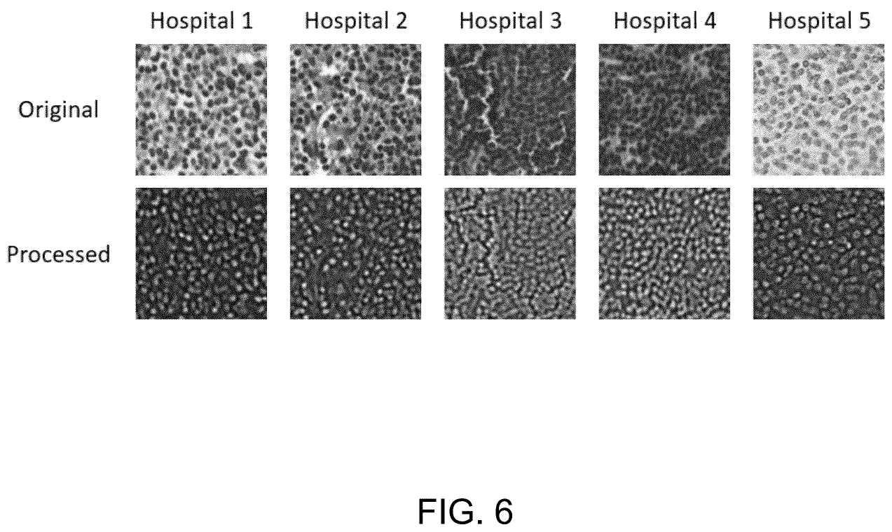

Histological images of breast cancer obtained from five distinct hospitals within the Camelyon17-WILDS dataset and corresponding refined images using PhyCV in accordance with an embodiment are illustrated in FIG. 6. The dataset comprises a total of 450,000 patches derived from 50 whole-slide-images (WSIs) of breast cancer metastases in lymph node sections. Specifically, 10 WSIs are sourced from each of the five hospitals located in the Netherlands. Each patch is of dimensions 96*96, with its label serving as a binary indicator denoting the presence of tumor tissue within the central 32*32 region.

In the example illustrated in FIG. 6, the training set includes 302,436 patches extracted from 30 WSIs, with 10 WSIs originating from each of the three hospitals designated for training. The validation set (In-Distribution) encompasses 33,560 patches derived from the same 30 WSIs utilized in the training set, while the validation set (Out-Of-Distribution) comprises 34,904 patches acquired from 10 WSIs belonging to the fourth hospital. Notably, these WSIs are distinct from those in the other splits. The test set (Out-Of-Distribution) comprises 85,054 patches obtained from 10 WSIs affiliated with the fifth hospital, chosen due to the unique visual distinctiveness of its patches.

Melanoma is a type of cancer that develops from the pigment-producing cells known as melanocytes. Melanocytes are the cells responsible for giving skin its color. While melanoma is substantially less common than other types of skin cancer, it is considered much more dangerous if it is not caught early, as it is more likely to spread to other parts of the body (metastasize). Diagnosing melanoma usually begins with a visual examination. If a mole or skin lesion appears suspicious, a biopsy (where a sample of the tissue is taken and analyzed) is performed. There are different types of biopsies depending on the size and location of the growth. One approach for melanoma detection in a point-of-care scenario is to utilize a cell phone camera, optionally enhanced using a macro lens attached to the phone's camera, to capture images of the lesion. Higher resolution images can be captured using a handheld microscope, for example, those from DinoLite, with optional polarization and fluorescent image capture capabilities. The image will then be classified as benign or malignant using an AI algorithm. While this is a promising solution, in practice, the efficacy is compromised by the inhomogeneity and variation in illumination, skin texture and color, and resolution of the imaging system. The data refinement techniques discussed in this document can be utilized to standardize images of melanoma to enable high-accuracy classification of the skin legion.

PhyCV in accordance with many embodiments can be applied to all applications of fluorescent imaging. Such applications include but are not limited to, Live Cell Tracking, Infectious Diseases Diagnostics, Protein Localization, Gene Expression, Cell Tracking, Enzyme Activity, Calcium Imaging and Brain Mapping, High-throughput Screening, and Surgical Guidance.

Immunohistochemistry (IHC) is a widely used technique in pathology and research that involves the staining of tissue sections with antibodies to detect specific antigens in the cells of a tissue sample. It combines anatomical, immunological, and biochemical techniques to identify discrete tissue components by the interaction of target antigens with specific antibodies tagged with a visible label. The fundamental principle behind IHC is antigen-antibody recognition—the ability of an antibody to specifically bind to a protein antigen in the tissue. The area where the antibody has bound can then be visualized under a microscope by various methods, depending on the type of label attached to the antibody. The efficacy and accuracy of IHC-based prognosis are limited by variabilities in the sample preparation and staining process, inhomogeneity in illumination, and resolution of the imaging system. This problem has been a significant barrier, restricting the accuracy of neural network classifiers and limiting the seamless integration of data and AI models across different platforms and institutions. Systems and methods in accordance with many embodiments can greatly enhance IHC's efficacy. For example, a particular antigen or biomarker on the tissue is identified by the color and intensity of a feature on the image. Color enhancement performed via computational imaging can enhance the prognosis efficacy.

The tumor microenvironment (TME) is the cellular environment containing tumor cells, immune cells, blood vessels, signaling molecules, and the extracellular matrix (ECM). The TME represents the frontline of the battleground between the tumor and the immune system. It is complex and dynamic, constantly evolving in response to the interactions between the tumor and the immune system. Its understanding and accurate characterization is a key to immunotherapy, one of the most promising approaches to cancer treatment. Systems and methods in accordance with many embodiments can advance the understanding of the tumor microenvironment for creating new immunotherapies, enhance diagnostic accuracy, reduce digital sample preparation and production time, and improve workflows through the refinement of data from digital pathology slides. It is a solution that has broad commercial applications in immunohistochemistry (IHC) and digital pathology.

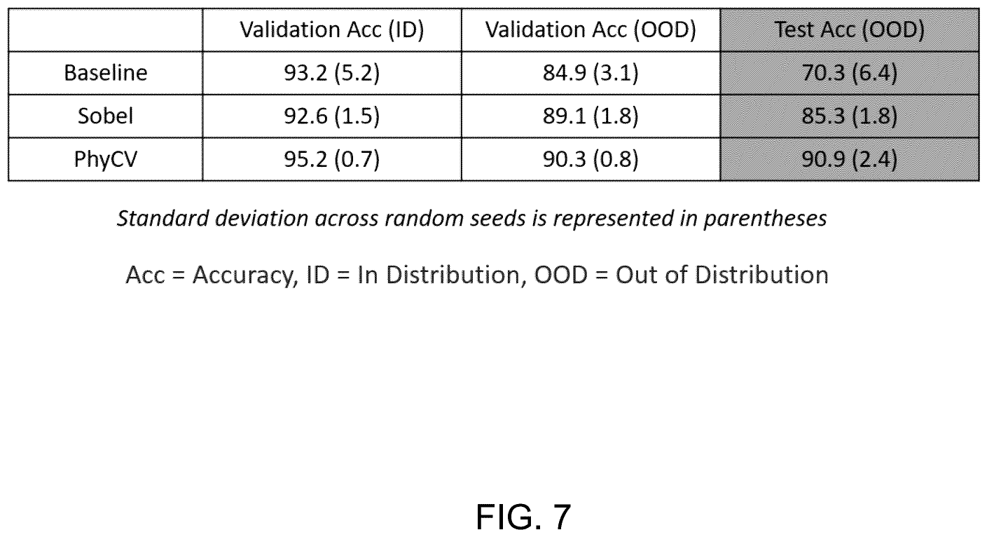

A comparison of neural network performance on the Camelyon17-WILDS dataset using various preprocessing techniques in accordance with an embodiment is illustrated in FIG. 7. The ‘Baseline’ condition involves no preprocessing of patches, while ‘Sobel’ signifies preprocessing with a Sobel gradient kernel during both training and inference. ‘PhyCV’ indicates preprocessing using PhyCV algorithms during both training and inference. The DenseNet-121 model is trained from scratch on the dataset with a learning rate of 10⁻³, L₂ regularization strength of 10⁻², a batch size of 32 and SGD with momentum (set to 0.9). Training comprises five epochs, and results for each preprocessing technique are aggregated over 10 random seeds, with standard deviation presented in parentheses.

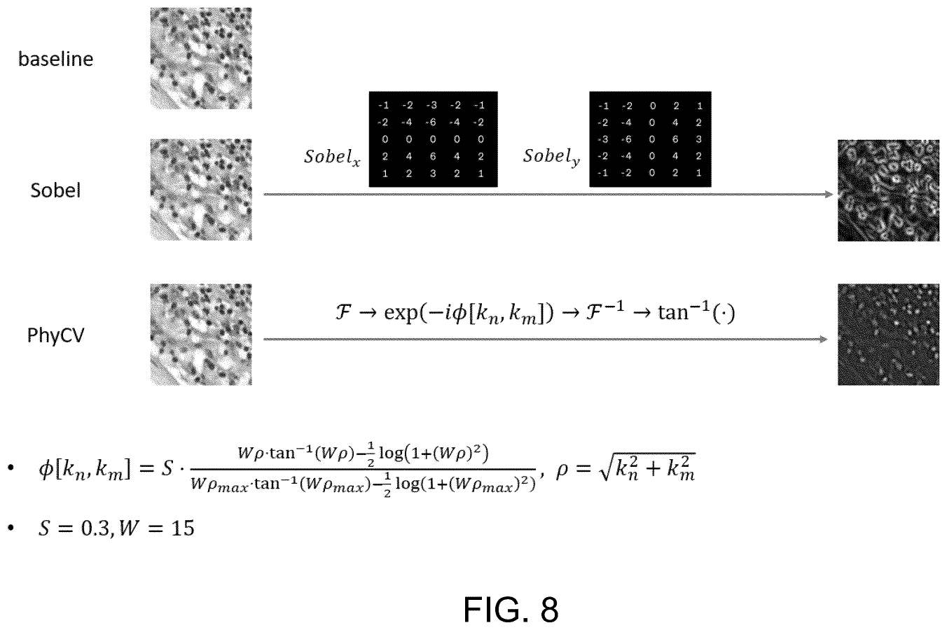

A comparison of neural network workflows between Sobel and PhyCV preprocessing in accordance with an embodiment is illustrated in FIG. 8. In Sobel, two convolutional kernels Sobelₓ and Sobel_{y} are applied to the original patch. In PhyCV, a kernel described in the polar coordinate system, as shown above, is applied to the spectrum of the image.

### Hardware Implementation

Processes that provide the methods and systems for preprocessing input data and performing inference tasks using preprocessed input data in accordance with some embodiments can be executed by a computing device or computing system, such as a desktop computer, tablet, mobile device, laptop computer, notebook computer, server system, and/or any other device capable of performing one or more features, functions, methods, and/or steps as described herein.

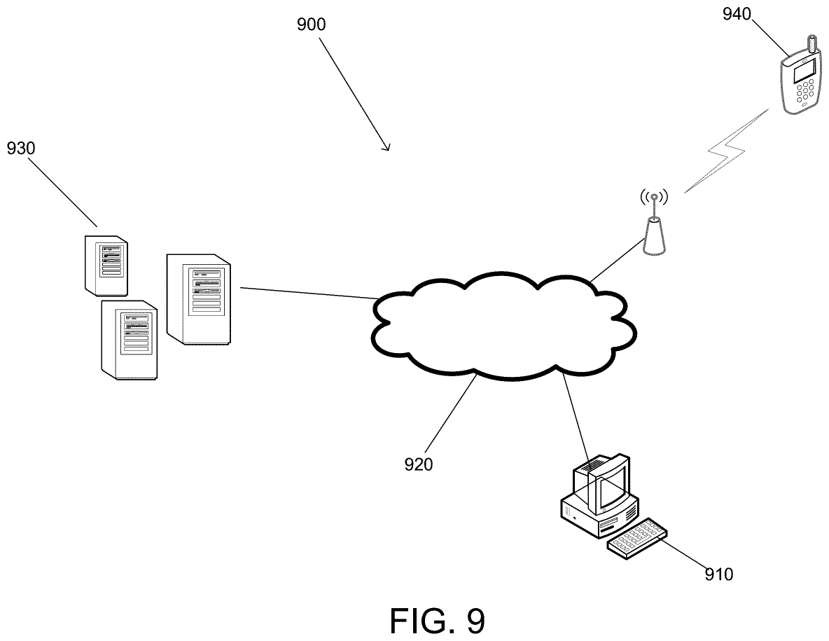

A network architecture for performing training and inference tasks using input data preprocessed by PhyCV in accordance with an embodiment of the invention is illustrated in FIG. 9. Such embodiments may be useful where computing power is not possible at a local level and a central computing device (e.g., server) performs one or more features, functions, methods, and/or steps described herein. In such embodiments, a computing device 910 (e.g., personal computer) is connected to a network 920 (wired and/or wireless), where it can receive inputs from one or more computing devices, including data from a records database or repository 930 containing video and/or image data for enhancing, data provided from a personal computing device, and/or any other relevant information from one or more other remote devices 910 and/or 940. Once computing device 910 performs one or more features, functions, methods, and/or steps described herein, any outputs can be transmitted to one or more computing devices 910 for entering into records.

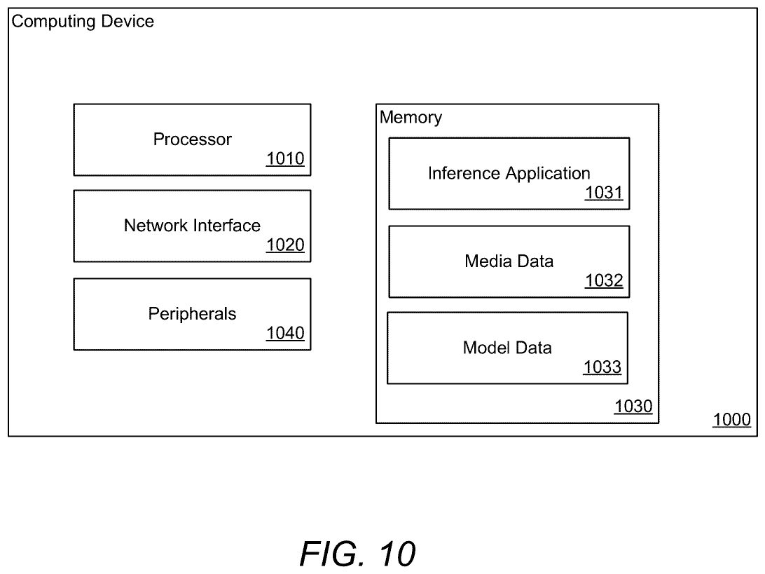

A computing device that can be utilized to perform training and inference tasks using input data preprocessed by PhyCV in accordance with an embodiment of the invention is illustrated in FIG. 10. Computing device 1000 includes a processor 1010. Processor 1010 may direct the inference application 1031 to perform training and inference tasks using input data preprocessed by PhyCV based on media data 1032 and model data 1033. In many embodiments, processor 1010 can include a processor, a microprocessor, a controller, or a combination of processors, microprocessor, and/or controllers that perform instructions stored in the memory 1030 to perform training and inference tasks using input data preprocessed by PhyCV. Processor instructions can configure the processor 1010 to perform processes in accordance with certain embodiments of the invention. In various embodiments, processor instructions can be stored on a non-transitory machine-readable medium. Computing device 1000 further includes a network interface 1020 that can receive media data from external sources. Computing device 1000 may further include a memory 1030 to store enhancement models under model data 1033. Computing device 1000 may further include peripherals 1040 to allow for user control and perform analysis of the enhancement process.

Although a specific example of a computing device is illustrated in this figure, any of a variety of computing devices can be utilized to perform training and inference tasks using input data preprocessed by PhyCV similar to those described herein as appropriate to the requirements of specific applications in accordance with embodiments of the invention.

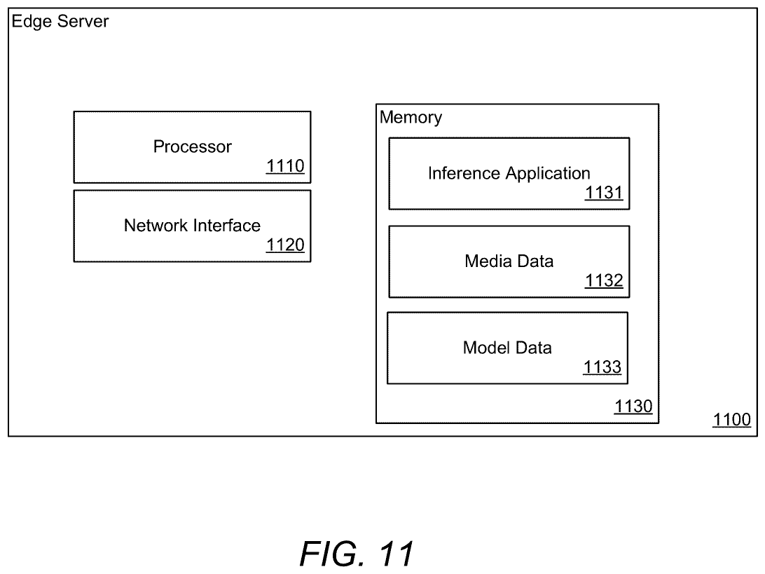

An edge server that can be utilized to perform training and inference tasks using input data preprocessed by PhyCV in accordance with an embodiment of the invention is illustrated in FIG. 11. Edge server 1100 includes a processor 1110. Processor 1110 may direct the inference application 1131 to perform training and inference tasks using input data preprocessed by PhyCV based on media data 1132 and model data 1133. In many embodiments, processor 1110 can include a processor, a microprocessor, a controller, or a combination of processors, microprocessors, and/or controllers that performs instructions stored in the memory 1130 to perform training and inference tasks using input data preprocessed by PhyCV. Processor instructions can configure the processor 1110 to perform processes in accordance with certain embodiments of the invention. In various embodiments, processor instructions can be stored on a non-transitory machine-readable medium. Edge server 1100 further includes a network interface 1120 that can receive media data from external sources. Edge server 1100 may further include a memory 1130 to store enhancement models under model data 1133. Computing device 1100 may further include peripherals 1140 to allow for user control and perform analysis of the enhancement process.

Although a specific example of an edge server is illustrated in this figure, any of a variety of edge servers can be utilized to perform training and inference tasks using input data preprocessed by PhyCV similar to those described herein as appropriate to the requirements of specific applications in accordance with embodiments of the invention.

In accordance with still other embodiments, the instructions for the processes can be stored in any of a variety of non-transitory machine-readable media appropriate to a specific application.

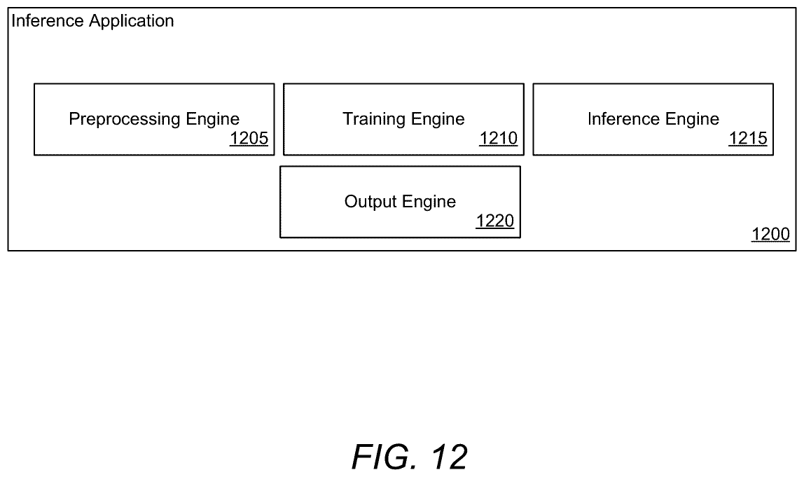

An inference application for performing inference tasks using input data preprocessed by PhyCV in accordance with an embodiment of the invention is illustrated in FIG. 12. Inference application 1200 includes Preprocessing engine 1205, training engine 1210, Inference engine 1215, and output engine 1220.

Preprocessing engines in accordance with a variety of embodiments can refine and transform input data to generate input with reduced non-semantic variability, thereby potentially improving learning efficiency and diagnostic accuracy.

In many embodiments, training engines can compute loss between generated predictions and actual labels and use the computed loss to inform the backpropagation of inference models that generated the predictions. Several embodiments update profiles of spectral phase kernels based on the training data and learning feedback, allowing for adaptive preprocessing.

Inference engines can generate predictions based on preprocessed input data. Output engines in accordance with several embodiments of the invention can provide a variety of outputs to a user, including (but not limited to) preprocessed inputs, generated predictions, diagnosis scores, confidence levels, notifications, and/or alerts. For example, output engines in accordance with various embodiments of the invention can provide a comparison of preprocessed input against the original inputs.

Although a specific example of an inference application is illustrated in FIG. 12, any of a variety of inference applications (e.g., with more or fewer modules) can be utilized to perform processes similar to those described herein as appropriate to the requirements of specific applications in accordance with embodiments of the invention.

Although specific methods of performing training and inference tasks using input data preprocessed by PhyCV are discussed above, many different methods of performing training and inference tasks using input data preprocessed by PhyCV can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for performing inference based on input data, the method comprising:
receiving a set of real-valued input images;
preprocessing the set of real-valued input images by applying a virtual optical dispersion to the set of real-valued input images to produce a set of real-valued output images;
predicting, using a machine learning model, an output based on the set of real-valued output images;
computing a loss based on the predicted output and a true output; and
updating the machine learning model based on the loss.

2. The method of claim 1, wherein preprocessing the set of real-valued input images comprises:
transforming a set of real-valued input images to a set of complex-valued input images;
applying a spectral phase kernel to the set of complex-valued input images;
converting the set of complex-valued input images to a set of real-valued intermediate output images;
determining an output phase based on the set of real-valued intermediate output images; and
generating a set of real-valued output images by modifying the set of real-valued intermediate output images with the determined output phase.

3. The method of claim 2, wherein transforming the set of real-valued input images comprises performing a 2D Fourier transform to project the real-valued input images into a spectral domain.

4. The method of claim 2, wherein applying a spectral phase kernel comprises multiplying the set of complex-valued input images by a complex exponential, wherein the argument of the complex exponential comprises:
a low-pass 2D function of frequency; and
a high-pass 2D function of frequency.

5. The method of claim 2, wherein converting the set of complex-valued input images comprises performing a 2D inverse Fourier transform to project the set of complex-valued input images back into the spatial domain.

6. The method of claim 2, wherein determining the output phase comprises computing an inverse tangent of the quotient of each pixel's imaginary component by each pixel's real component for the set of complex-valued input images.

7. The method of claim 2, wherein the output phase is determined using a Fourier differential theorem to approximate the output phase.

8. The method of claim 2, wherein the set of real-valued input images comprises pathology images for cancer detection and tumor microenvironment analysis.

9. The method of claim 2, wherein the spectral phase kernel comprises a Phase Stretch Transform (PST) algorithm for feature extraction and image processing in medical imaging applications.

10. The method of claim 9 wherein the PST algorithm preprocesses input images in the spatial domain by utilizing Fourier differentiation property.

11. The method of claim 1, wherein the preprocessing comprises a Vision Enhancement via Virtual Diffraction and coherent Detection (VEViD) method for low-light enhancement and color-enhancement.

12. The method of claim 1, further comprising updating the preprocessing based on the computed loss.

13. The method of claim 1, wherein the machine learning model comprises a convolutional neural network and a vision transformer.

14. The method of claim 2, wherein the set of real-valued input images are transformed into HSV (hue, saturation, value) color space.

15. The method of claim 2, wherein the output phase indicates dispersion effects of the spectral phase kernel on the set of real-valued input images.

16. A method for preprocessing input data, the method comprising:
adding a constant DC bias term to each pixel of an input image to create a first modified image;
multiplying each pixel of the first modified image by a negative constant gain value to create a second modified image;
dividing each pixel of the second modified image by a spatially corresponding pixel of the input image to create a third modified image; and
obtaining an output image by computing the inverse tangent of each pixel of the third modified image.

17. The method of claim 16, further comprising normalizing the output image by removing the DC component from the image data and equalizing the image.
